(19) United States
(12) Patent Application Publication
(10) Pub. No.: US 2016/0054276 A1
(43) Pub. Date: Feb. 25, 2016

(54) METHOD FOR ESTABLISHING SHENQI FUZHENG INJECTION FINGERPRINT SPECTRUM

(71) Applicant: LI MIN PHARMACEUTICAL FACTORY OF LIVZON PHARMACEUT ICAL GROUP, Shaoguan City, Guangdong (CN)

(72) Inventors: Yangang SONG, Guangdong (CN); Xuehua LIU, Guangdong (CN); Wenhua HUANG, Guangdong (CN); Donglai LIU, Guangdong (CN)

(73) Assignee: LI MIN PHARMACEUTICAL FACTORY OF LIVZON PHARMACEUT ICAL GROUP, Shaoguan City, Guangdong (CH)

(21) Appl. No.: 14/439,635

(22) PCT Filed: Nov. 1, 2013
(86) PCT No.: PCT/CN2013/086405

§ 371 Date: Jul. 13, 2015

(30) Foreign Application Priority Data
Nov. 2, 2012 (CN) 201210436493.4

(51) Int. Cl. G01N 30/86; G01N 33/15; G01N 30/72

(52) U.S. Cl. CPC G01N 30/8686; G01N 30/7233; G01N 33/15

(57) ABSTRACT

A method for establishing a Shenqi Fuzheng injection fingerprint spectrum, comprising: employing an ultra-high voltage liquid chromatography mass spectrometer to test the Shenqi Fuzheng injection, the chromatography conditions including: chromatographic column: Agilent Zorbax Eclipse Plus C18, 2.1 mm×100 mm, 1.8 μm; mobile phase: mobile phase A is 0.1% formic acid aqueous solution, and mobile phase B is 0.1% formic acid acetonitrile solution; employing gradient elution procedure as follows: 0-0.5 min, 95% of mobile phase A, and 5% of mobile phase B; 0.5-10 min, 95%-75% of mobile phase A, and 5%-25% of mobile phase B; 10-15 min, 75%-45% of mobile phase A, and 25%-55% of mobile phase B; 15-18 min, 45%-0% of mobile phase A, and 55%-100% of mobile phase B; and 18-20 min, 0% of mobile phase A, and 100% of mobile phase B.

### FIELD OF THE INVENTION

The present invention belongs to the field of drug testing, particularly relates to a method for establishing a Shenqi Fuzheng injection fingerprint profile.

### BACKGROUND OF THE INVENTION

Quality control of traditional Chinese herbal compound is one of the critical problems restricting development in modernization of traditional Chinese medicine. The basic theory of traditional Chinese medicine emphasizes the overall effect of the medicines and attaches importance to the synergistic effects on efficacy. Taking one or two effective components in Chinese medicines as qualitative and quantitative indexes is far from effectively controlling and assessing the quality of Chinese medicines, more difficult to reflect the safety and effectiveness of Chinese medicines. Traditional Chinese herbal compound is a compound preparation, which combines more than two kinds of Chinese medicinal herbs to be used in disease treatment, whose quality control is more difficult than single Chinese medicinal herb. In recent years, Chinese medicine fingerprint profile and characteristic fingerprint profile are widely used in quality control, wherein the Chinese medicine characteristic fingerprint profile refers to a characteristic fingerprint profile made up by selecting several chromatographic peaks with good specificity or a combination of chromatographic peaks with good specificity from the Chinese medicine fingerprint profile, which can be used to monitor the quality of Chinese medicines by observing the presence or absence and changes of the characteristic fingerprint peaks. In Chinese Pharmacopoeia, the 2010 Edition, the characteristic fingerprint profile has been widely applied in quality control of intermediate components of various Chinese medicine.

Ultra high performance liquid chromatography (UHPLC) technology is a great breakthrough of the chromatography technique. It has advantages in ultra-high speed, ultra-high sensitivity and ultra-high resolution, and it achieves faster and more sensitive detection performance by using a smaller chromatographic column packing technology. It is widely used in pesticide residues and drug metabolism in foreign countries, and its application become more and more common in China.

Chromatography-mass spectrometry technology is an advanced analysis technique developing rapidly in recent years, wherein, after extensive application of the gas chromatography-mass spectrometry (GC-MS) technology, the liquid chromatography-mass spectrometry (LC-MS) technology is an another technique which is gradually recognized and accepted, but it is not yet widely applied because of expensive equipment. The ionization technology adopted in liquid chromatography-mass spectrometers can not only resolve the problems in detecting some ingredients without ultraviolet absorption, such as saponins, but also get a precise molecular weight of the ionized component, which provides data support for identification and confirmation of the component and the structure thereof.

Shenqi Fuzheng injection is a Chinese medicine infusion, with effect of benefiting qi for strengthening resistance, and is used for the treatment of lassitude, shortness of breath with no desire to speak, spontaneous sweating and vertigo caused by lung-spleen deficiency, and used as an adjuvant therapy for lung cancer and gastric cancer patients with the syndromes above. It is one of National Protection Varieties of Traditional Chinese Medicine, and the Protection Variety No. is: ZYB2072004073. Among quality standards for Shenqi Fuzheng injection, the “Fingerprint Profile” item saying detection by using high performance liquid chromatography-ultraviolet detector has some limitations, for example, the main component saponin has no ultraviolet absorption. The component saponin may be monitored by using high performance liquid chromatography-evaporative light scattering detector, but pre-treatments for samples are complicated and furthermore the analysis takes a long time. Therefore, the main components cannot be monitored comprehensively and quickly, and there needs to be improved.

### SUMMARY OF THE INVENTION

The technical problem to be solved in the present invention is to remedy deficiencies in prior art, and the objective of the present invention is to provide a method for establishing Shenqi Fuzheng injection fingerprint profile. The fingerprint profile established by the method described in the present invention can be used as a standard fingerprint profile to be applied in identification of Shenqi Fuzheng injection.

The present invention achieves the above objective by use of the following technical solutions:

A method for establishing ShengQI FuZheng injection fingerprint profile, wherein the method comprises testing Shenqi Fuzheng injection by ultra-high pressure liquid chromatography-mass spectrometer, for example ultra-high performance liquid chromatography-quadrupole time-of-flight mass spectrometer, wherein the chromatographic conditions include the followings:
Chromatographic column: Agilent Zorbax Eclipse Plus C18, 2.1 mm×100 mm, 1.8 μm;
Mobile phase: Mobile phase A is 0.1% (v/v) formic acid aqueous solution, mobile phase B is 0.1% (v/v) formic acid acetonitrile solution;
Using gradient elution according to the following elution program, wherein the proportions of the mobile phases are all volume percentages:
0-5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%.

Preferably, the chromatographic conditions further include the followings:
Flow rate: 0.35 ml/min;
Column temperature: 40° C.;
Injection volume: 5 μl.

Preferably, in the method described above for establishing ShengQI FuZheng injection fingerprint profile, the mass spectrometry conditions include the followings:

The ion source is an ESI source, and detection is operated in negative ion mode;
Atomized gas pressure: 35 psig;
Dry gas temperature: 350° C.;
Dry gas flow rate: 10 L/min;
Capillary voltage: 3,500 V;
Voltage at capillary exit: 135 V.

Preferably, the method described above for establishing Shenqi Fuzheng injection fingerprint profile further comprises preparation of control solutions by following steps:

Accurately weighing an appropriate amount of calycosin glucoside or astragaloside IV, and then adding methanol to prepare a solution containing 0.004 mg of calycosin glucoside per ml or 0.006 mg of astragaloside IV per ml, respectively.

Preferably, the method described above for establishing ShengQI FuZheng injection fingerprint profile further comprises the preparation of test sample solution by the following step: filtering the Shenqi Fuzheng injection through a 0.22 μm microporous filter membrane.

Preferably, the method described above for establishing ShengQI FuZheng injection fingerprint profile comprises the following steps:
(1) Preparation of control solution: Accurately weighing an appropriate amount of calycosin glucoside or astragaloside IV, then adding methanol to prepare a solution containing 0.004 mg of calycosin glucoside per ml or 0.006 mg of astragaloside IV per ml, respectively;
(2) Preparation of test sample solution: Filtering Shenqi Fuzheng injection through a 0.22 μm microporous filter membrane;
(3) Determination: Accurately aspirating 5 μl of the control solution or the test sample solution, respectively, and then injecting the solutions into a ultra-high pressure liquid chromatography-mass spectrometer, for example ultra-high performance liquid chromatography-quadrupole time-of-flight mass spectrometer, conducting determination according to the following conditions to obtain the Shenqi Fuzheng injection fingerprint profile:
wherein the chromatographic conditions include the followings:
Chromatographic column: Agilent Zorbax Eclipse Plus C18, 2.1 mm×100 mm, 1.8 μm;
Mobile phase: mobile phase A is 0.1% (v/v) formic acid aqueous solution, mobile phase B is 0.1% (v/v) formic acid acetonitrile solution;
Using gradient elution according to the following elution program, wherein the proportions of the mobile phases are all volume percentages:
0-5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%;

Preferably, the chromatographic conditions further include the followings:
Flow rate: 0.35 ml/min;
Column temperature: 40° C.;

Preferably, the mass spectrometry conditions include the followings:
The ion source is an ESI source, and detection is operated in negative ion mode;
Atomized gas pressure: 35 psig;
Dry gas temperature: 350° C.;
Dry gas flow rate: 10 L/min;
Capillary voltage: 3,500 V;
Voltage at capillary exit: 135 V.

Preferably, the method described above for establishing Shenqi Fuzheng injection fingerprint profile further comprises:

Comparing multiple Shenqi Fuzheng injection fingerprint profiles, picking out common characteristic peaks to obtain the Shenqi Fuzheng injection characteristic fingerprint profile.

Preferably, in the method described above for establishing Shenqi Fuzheng injection fingerprint profile, the Shenqi Fuzheng injection fingerprint profile or Shenqi Fuzheng injection characteristic fingerprint profile comprises 18 characteristic peaks, the retention time of each characteristic peak is as follows:

Peak 1: 7.1 min, Peak 2: 7.5 min, Peak 3: 8.1 min, Peak 4: 8.6 min, Peak 5: 9.2 min, Peak 6: 9.9 min, Peak 7: 10.9 min, Peak 8: 11.3 min, Peak 9: 11.7 min, Peak 10: 12.7 min, Peak 11: 13.4 min, Peak 12: 13.7 min, Peak 13: 14.4 min, Peak 14: 14.8 min, Peak 15: 15.1 min, Peak 16: 15.5 min, Peak 17: 15.9 min, Peak 18: 16.3 min.

Preferably, in the method described above for establishing Shenqi Fuzheng injection fingerprint profile, the Shenqi Fuzheng injection fingerprint profile or the Shenqi Fuzheng injection characteristic fingerprint profile takes the control astragaloside IV as a reference peak, by which the relative retention time of each characteristic peak is calculated, as follows:

Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, Peak 18: 1.19.

Preferably, in the Shenqi Fuzheng injection fingerprint profile or the Shenqi Fuzheng injection characteristic fingerprint profile, Peak 2 and Peak 12 are calycosin glucoside and astragaloside IV, respectively; preferably, the ratio of the area of calycosin glucoside peak and the astragaloside IV peak to the area of the corresponding reference peak is 0.5-1.5.

The present invention also provide a method for identifying Shenqi Fuzheng injection, wherein the method comprises comparing the fingerprint profile or the characteristic fingerprint profile of the test sample established according to the method described above with the standard fingerprint profile or the characteristic fingerprint profile established according to the method described above so as to identify authenticity.

In a preferred embodiment, the present invention provide a method for establishing Shenqi Fuzheng injection fingerprint profile, wherein the method comprises the following steps:
Preparing a mixed control solution of calycosin glucoside and astragaloside IV, containing calycosin glucoside at a concentration of 0.004 mg/ml and astragaloside IV at a concentration of 0.006 mg/ml;
Taking a filtrate of Shenqi Fuzheng injection as a test solution;
Analyzing the control solution and the test sample solution described above by ultra high performance liquid chromatography-quadrupole time-of-flight mass spectrometer, wherein the chromatographic conditions include the followings:
Chromatographic column: Agilent Zorbax Eclipse Plus C18, 2.1 mm×100 mm, 1.8 μm;
Mobile phase: mobile phase A is 0.1% (v/v) formic acid aqueous solution, mobile phase B is 0.1% (v/v) formic acid acetonitrile solution;
Gradient elution;
Flow rate: 0.35 ml/min;
Column temperature: 40° C.;
Injection volume: 5 μl;
Ultra high performance liquid chromatography-quadrupole time-of-flight mass spectrometry fingerprint profile (total ion chromatogram profile) for Shenqi Fuzheng injection is obtained;
The steps of gradient elution include the followings: 0-5 min, mobile phase acetonitrile-water is 5:95; 0.5-10 min, mobile phase acetonitrile-water changes from 5:95 to 25:75 gradually; 10-15 min, mobile phase acetonitrile-water changes from 25:75 to 55:45 gradually; 15-18 min, mobile phase acetonitrile-water changed from 55:45 to 100:0 gradually; 18-20 min, mobile phase acetonitrile-water is 100:0.

Shenqi Fuzheng injection characteristic fingerprint profile is determined according to the fingerprint profile (total ion chromatogram profile) and the characteristic fingerprint profile (extracted ion chromatogram profile) extracted from the fingerprint profile (total ion chromatogram profile), and thereby monitoring the quality of Shenqi Fuzheng injection.

The gradient elution steps can also be shown in Table 1:

TABLE 1
Gradient Elution Program

| Time (min) | Mobile Phase A (%) | Mobile Phase B (%) |
|---|---|---|
| 0-0.5 | 95 | 5 |
| 0.5-10 | 95 → 75 | 5 → 25 |
| 10-15 | 75 → 45 | 25 → 55 |
| 15-18 | 45 → 0 | 55 → 100 |
| 18-20 | 0 | 100 |

According to the method in the present invention, total ion chromatograms (TIC) of 100 batches of Shenqi Fuzheng injection are analyzed and compared to sorted out the common characteristic peaks, the ion mass numbers of which are used to obtain the extracted ion chromatograms (EIC), followed by marking the retention time (Rt) of each common characteristic peak, so as to obtain the Shenqi Fuzheng injection characteristic fingerprint profile.

There are 18 common characteristic peaks, with a retention time (Rt) and a mass number as follows: 7.1 min (471.2083), 7.5 min (491.1195), 8.1 min (441.1919), 8.6 min (309.1555), 9.2 min (187.0976), 9.9 min (441.1766), 10.9 min (593.1876), 11.3 min (507.1508), 11.7 min (463.1610), 12.7 min (991.5119), 13.4 min (991.5119), 13.7 min (829.4591), 14.4 min (871.4697), 14.8 min (871.4697), 15.1 min (871.4697), 15.5 min (913.4650), 15.9 min (913.4650), 16.3 min (913.4650), respectively, wherein the chromatographic peaks with a Rt of 7.5 min and 13.7 min are verified as calycosin glucoside and astragaloside IV, respectively; Peak S is a peak corresponding to the astragaloside IV reference peak; the relative retention time of each characteristic peak is calculated, wherein the retention time should fluctuate within ±5% of the specified values, which are listed in an order of 0.52, 0.54, 0.59, 0.62, 0.66, 0.72, 0.79, 0.82, 0.85, 0.92, 0.97, 1.00, 1.04, 1.07, 1.10, 1.13, 1.16, 1.19; wherein the ratio of the peak area of calycosin glucoside and astragaloside IV to the peak area of their corresponding reference should be within 0.5-1.5.

The characteristic fingerprint profile established by the method of the present invention can be used in identifying Shenqi Fuzheng injection.

Compared with the prior art, the present invention has beneficial effects as follows:

In the quality standards for Shenqi Fuzheng injection, the “Fingerprint Profile” item says that Shenqi Fuzheng injection fingerprint profile is determined by high performance liquid chromatography-UV detection, which achieves the objective of monitoring the quality to a certain extent, but the main component saponins substantially has no absorption under ultraviolet; the “Content Determination” item says that the content of total saponins is determined by UV spectrophotometry with vanillin-glacial acetic acid, and the content of astragaloside IV is determined by high performance liquid chromatography-evaporative light scattering detection, but these still cannot fully reflect the content of each saponin component. In the internal control in enterprise, the saponin component can be monitored by further using high performance liquid chromatography-evaporative light scattering detector to determine the fingerprint profile, but pre-treatments for samples are required additionally, and the analysis would take a long time.

Therefore, the technical standards for Shenqi Fuzheng injection characteristic fingerprint profile established by ultra high performance liquid chromatography-quadrupole time-of-flight mass spectrometry according to the method of the present invention can make it easier to monitor drug quality comprehensively, quickly, and effectively, depending on presence or absence and characteristics of common peaks in the characteristic fingerprint profile, so as to ensure stable, uniform and controllable qualities. The present invention has features in is advanced method and better stability and reproducibility.

### BRIEF DESCRIPTION OF THE DRAWINGS

FIG. 1 illustrates a total ion chromatogram (TIC) of the Shenqi Fuzheng injection provided by the present invention, wherein the arrows from left to right indicate the characteristic peaks 1 to 18, respectively;

FIG. 2 illustrates an extracted ion chromatogram (EIC) of the Shenqi Fuzheng injection, wherein the arrows from left to right indicate the characteristic peaks 1 to 18, respectively;

FIG. 3 illustrates an extracted ion chromatogram (EIC) of the control mixture, wherein Peaks 2 and 12 represent calycosin glucoside and astragaloside IV, sequentially.

FIG. 4 is a graph illustrating the comparison of the Shenqi Fuzheng injection fingerprint profile of the present invention with a counterfeit, wherein “1” showing the certified Shenqi Fuzheng injection, “2” showing the counterfeit (presumed as Danshen Injection), and “3” showing a Danshen infusion solution.

### DETAILED DESCRIPTION OF THE EMBODIMENTS

The technical solutions of the present invention will be further described in details in combination with specific examples.

### Example 1

### 1. Instrument and Test Drug

1.1 Instruments: Agilent LC/MSD (1290UHPLC dual-gradient pump, built-in vacuum degasser, 100-bit automatic sampler, intelligent column oven, high-precision quadrupole tandem time-of-flight mass spectrometer system); Chromatographic column: Agilent Zorbax Eclipse Plus C18 (2.1 mm×100 mm, 1.8 μm).

1.2 Test drug: Shenqi Fuzheng injection, provided by Livzon Group Limin Pharmaceutical Factory. Reagents acetonitrile and formic acid used in the experiments were both chromatographically pure, and the water was ultrapure water.

### 2. Method and result

2.1 Preparation of test sample solution: Shenqi Fuzheng injection was filtered through a 0.22 μm microporous filter membrane.

2.2 Preparation of mixed control solution: An appropriate amount of calycosin glucoside and astragaloside IV was accurately weighed, and methanol was then added to prepare a solution containing 0.004 mg of calycosin glucoside and 0.006 mg of astragaloside IV per ml, respectively.

2.3 Chromatographic conditions: Chromatographic column was Agilent Zorbax Eclipse Plus C18, 2.1 mm×100 mm, 1.8 μm; Mobile phases were 0.1% formic acid aqueous solution (A) and 0.1% formic acid acetonitrile solution (B); Column temperature was 40° C.; Injection volume was 5 μl; A gradient elution program as shown in Table 2 was used:

TABLE 2
Gradient Elution Program

| Time (min) | Mobile Phase A (%) | Mobile Phase B (%) |
|---|---|---|
| 0-0.5 | 95 | 5 |
| 0.5-10 | 95 → 75 | 5 → 25 |
| 10-15 | 75 → 45 | 25 → 55 |
| 15-18 | 45 → 0 | 55 → 100 |
| 18-20 | 0 | 100 |

2.4 Mass spectrometry conditions: The ion source was an ESI source, detection was operated in negative ion mode; Atomized gas pressure: 35 psig; Dry gas temperature: 350° C.; Dry gas flow rate: 10 L/min; Vcap Capillary voltage: 3,500 V; Voltage at capillary exit: 135 V.

2.5 Determination Method

5 μl of the control solution or the test sample solution was accurately aspirated, respectively, then injected into the liquid chromatography-mass spectrometer, respectively, determination was conducted, recording the spectra for 20 minutes.

2.6 Determination of Common Characteristic Peaks

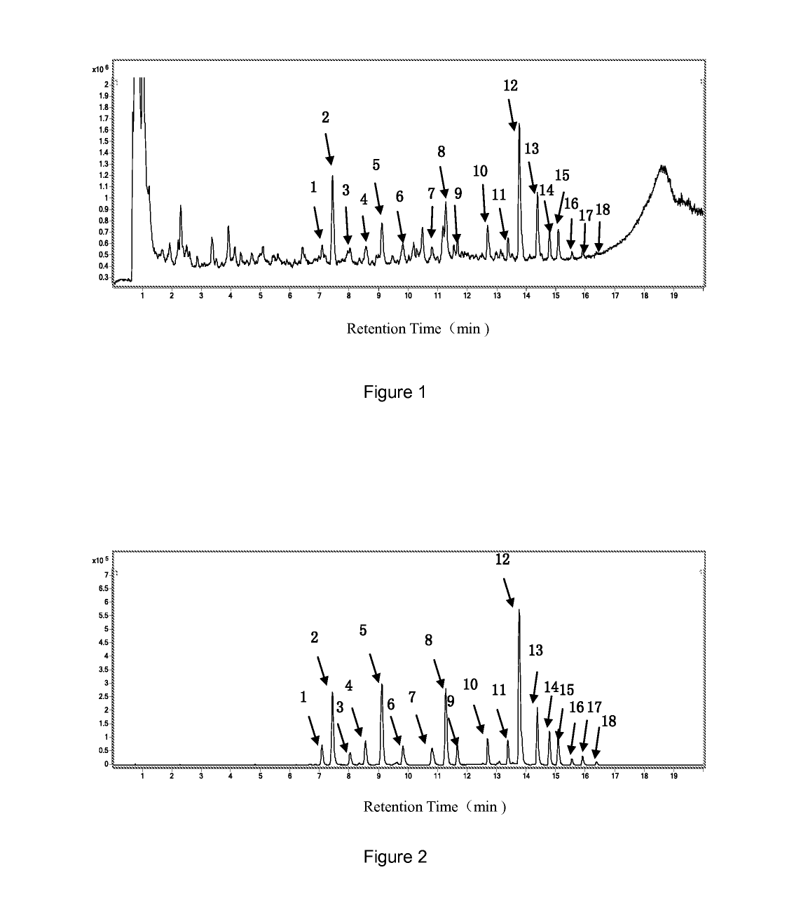

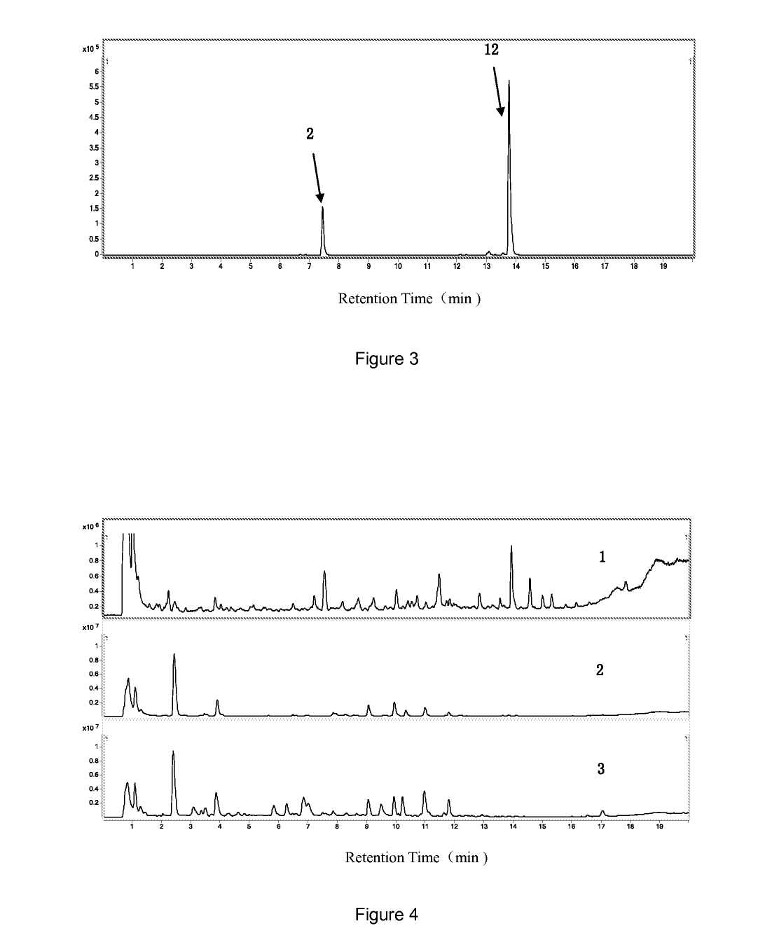

The common characteristic peaks were sorted out by comparing the total ion chromatograms (TIC) of 100 batches of Shenqi Fuzheng injection, see details in FIG. 1; the extracted ion chromatogram (EIC) was obtained by using the ion mass number of these common characteristic peaks, see details in FIG. 2 (specifically, a series of target ions were extracted from FIG. 1 by using the qualitative analysis software in the data analysis software Masshunter and utilizing the function of ion extraction, and thereby obtaining FIG. 2); then the retention time (Rt) of each common characteristic peak was marked, so as to obtain the Shenqi Fuzheng injection characteristic fingerprint profile. There were 18 common characteristic peaks, with a retention time (Rt) and a mess number as follows: 7.1 min (471.2083), 7.5 min (491.1195), 8.1 min (441.1919), 8.6 min (309.1555), 9.2 min (187.0976), 9.9 min (441.1766), 10.9 min (593.1876), 11.3 min (507.1508), 11.7 min (463.1610), 12.7 min (991.5119), 13.4 min (991.5119), 13.7 min (829.4591), 14.4 min (871.4697), 14.8 min (871.4697), 15.1 min (871.4697), 15.5 min (913.4650), 15.9 min (913.4650), 16.3 min (913.4650), wherein the chromatographic peaks with a Rt of 7.5 min and 13.7 min were verified as calycosin glucoside and astragaloside IV respectively, see details in FIG. 3; Peak S was a peak corresponding to an astragaloside IV reference peak, the retention time of each characteristic peaks was calculated, wherein the retention time should fluctuate within ±5% of the specified values, which were listed in an order of 0.52, 0.54, 0.59, 0.62, 0.66, 0.72, 0.79, 0.82, 0.85, 0.92, 0.97, 1.00, 1.04, 1.07, 1.10, 1.13, 1.16, 1.19; the ratio of the peak area of calycosin glucoside and astragaloside IV to the peak area of their corresponding reference should be within 0.5-1.5.

2.7 Precision assay: The test sample solution from the same Shenqi Fuzheng injection was injected for continuously 6 times, followed by extracting the characteristic peaks and marking the retention time. Peak 12 of astragaloside IV was taken as Peak S, and then the relative retention time of each of other characteristic peaks were calculated. The results showed that the RSD values of the relative retention time of each characteristic peak was all less than 1%, and the precision of the instrument was excellent. The results of the precision assay are shown in Table 3:

TABLE 3
Results of Precision Assay

| | 1 | 2 | 3 | 4 | 5 | 6 | RSD (%) |
|---|---|---|---|---|---|---|---|
| 1 | 0.517 | 0.517 | 0.517 | 0.516 | 0.516 | 0.517 | 0.14 |
| 2 | 0.542 | 0.542 | 0.542 | 0.541 | 0.541 | 0.542 | 0.09 |
| 3 | 0.586 | 0.586 | 0.586 | 0.585 | 0.585 | 0.586 | 0.08 |
| 4 | 0.623 | 0.624 | 0.624 | 0.623 | 0.623 | 0.624 | 0.08 |
| 5 | 0.661 | 0.661 | 0.661 | 0.661 | 0.661 | 0.661 | 0.04 |
| 6 | 0.716 | 0.715 | 0.716 | 0.715 | 0.716 | 0.716 | 0.06 |
| 7 | 0.786 | 0.786 | 0.787 | 0.787 | 0.788 | 0.787 | 0.08 |
| 8 | 0.820 | 0.819 | 0.820 | 0.820 | 0.820 | 0.821 | 0.08 |
| 9 | 0.847 | 0.847 | 0.848 | 0.848 | 0.849 | 0.848 | 0.06 |
| 10 | 0.923 | 0.922 | 0.923 | 0.922 | 0.923 | 0.923 | 0.01 |
| 11 | 0.972 | 0.972 | 0.972 | 0.972 | 0.972 | 0.972 | 0.02 |
| S | 1.000 | 1.000 | 1.000 | 1.000 | 1.000 | 1.000 | 0.00 |
| 13 | 1.044 | 1.044 | 1.044 | 1.045 | 1.044 | 1.044 | 0.02 |
| 14 | 1.074 | 1.074 | 1.074 | 1.074 | 1.074 | 1.074 | 0.02 |
| 15 | 1.096 | 1.096 | 1.096 | 1.096 | 1.096 | 1.096 | 0.01 |
| 16 | 1.129 | 1.129 | 1.128 | 1.129 | 1.129 | 1.129 | 0.03 |
| 17 | 1.155 | 1.155 | 1.155 | 1.156 | 1.156 | 1.155 | 0.02 |
| 18 | 1.189 | 1.189 | 1.189 | 1.189 | 1.189 | 1.189 | 0.02 |

2.8 Stability assay: The test sample solution from the same Shenqi Fuzheng injection was injected at 0 hour, at 1 hour, at 2 hours, at 4 hours, at 8 hours, and at 12 hours, respectively, followed by extracting the characteristic peaks and marking the retention time. Peak 12 of astragaloside IV was taken as Peak S, and then the relative retention time of each of other characteristic peaks was calculated. The results showed that the RSD values of the relative retention time of each characteristic peak was all less than 1%, and the test sample solution remained stable within 12 hours during standing. The results of the stability assay are shown in Table 4:

TABLE 4
Results of Stability Assay

| | 0 | 1 | 2 | 4 | 8 | 12 | RSD (%) |
|---|---|---|---|---|---|---|---|
| 1 | 0.517 | 0.516 | 0.516 | 0.517 | 0.517 | 0.517 | 0.15 |
| 2 | 0.542 | 0.541 | 0.541 | 0.542 | 0.541 | 0.543 | 0.11 |
| 3 | 0.586 | 0.586 | 0.585 | 0.586 | 0.586 | 0.587 | 0.09 |
| 4 | 0.624 | 0.623 | 0.623 | 0.624 | 0.623 | 0.624 | 0.09 |
| 5 | 0.662 | 0.662 | 0.661 | 0.662 | 0.661 | 0.662 | 0.05 |
| 6 | 0.716 | 0.716 | 0.715 | 0.716 | 0.715 | 0.716 | 0.06 |
| 7 | 0.787 | 0.786 | 0.786 | 0.788 | 0.787 | 0.786 | 0.07 |
| 8 | 0.820 | 0.820 | 0.819 | 0.820 | 0.820 | 0.821 | 0.04 |
| 9 | 0.848 | 0.848 | 0.847 | 0.848 | 0.848 | 0.847 | 0.04 |
| 10 | 0.922 | 0.922 | 0.923 | 0.923 | 0.922 | 0.923 | 0.02 |
| 11 | 0.972 | 0.972 | 0.972 | 0.972 | 0.972 | 0.972 | 0.01 |
| S | 1.000 | 1.000 | 1.000 | 1.000 | 1.000 | 1.000 | 0.00 |
| 13 | 1.044 | 1.044 | 1.044 | 1.044 | 1.044 | 1.045 | 0.01 |
| 14 | 1.074 | 1.074 | 1.074 | 1.074 | 1.074 | 1.074 | 0.01 |
| 15 | 1.096 | 1.096 | 1.096 | 1.096 | 1.096 | 1.096 | 0.02 |
| 16 | 1.129 | 1.129 | 1.129 | 1.129 | 1.129 | 1.129 | 0.02 |
| 17 | 1.155 | 1.156 | 1.156 | 1.155 | 1.156 | 1.156 | 0.02 |
| 18 | 1.189 | 1.190 | 1.189 | 1.189 | 1.189 | 1.190 | 0.04 |

2.9 Repeatability assay: Six Shenqi Fuzheng injection of the same batch were taken, prepared according to the method of preparing the test sample solution, and then injected, respectively, followed by extracting the characteristic peaks and marking the retention time. Peak 12 of astragaloside IV was taken as peak S, and then the relative retention time of each of other characteristic peaks was calculated. The results showed that the RSD values of the relative retention time of each characteristic peak was all less than 1%, and the method has good repeatability. The results of the repeatability assay are shown in Table 5:

TABLE 5
Results of Repeatability Test

| | 1 | 2 | 3 | 4 | 5 | 6 | RSD (%) |
|---|---|---|---|---|---|---|---|
| 1 | 0.512 | 0.511 | 0.511 | 0.512 | 0.511 | 0.517 | 0.45 |
| 2 | 0.537 | 0.536 | 0.536 | 0.536 | 0.536 | 0.542 | 0.45 |
| 3 | 0.581 | 0.581 | 0.580 | 0.580 | 0.581 | 0.586 | 0.40 |
| 4 | 0.619 | 0.619 | 0.618 | 0.619 | 0.618 | 0.624 | 0.38 |
| 5 | 0.655 | 0.655 | 0.655 | 0.655 | 0.655 | 0.661 | 0.37 |
| 6 | 0.711 | 0.710 | 0.710 | 0.710 | 0.709 | 0.716 | 0.36 |
| 7 | 0.782 | 0.782 | 0.781 | 0.782 | 0.781 | 0.786 | 0.25 |
| 8 | 0.815 | 0.815 | 0.815 | 0.815 | 0.815 | 0.820 | 0.25 |
| 9 | 0.843 | 0.844 | 0.844 | 0.844 | 0.844 | 0.848 | 0.18 |
| 10 | 0.922 | 0.922 | 0.922 | 0.922 | 0.922 | 0.923 | 0.03 |
| 11 | 0.972 | 0.972 | 0.972 | 0.972 | 0.972 | 0.972 | 0.01 |
| S | 1.000 | 1.000 | 1.000 | 1.000 | 1.000 | 1.000 | 0.00 |
| 13 | 1.045 | 1.044 | 1.045 | 1.044 | 1.044 | 1.044 | 0.02 |
| 14 | 1.074 | 1.075 | 1.075 | 1.075 | 1.074 | 1.074 | 0.03 |
| 15 | 1.096 | 1.097 | 1.097 | 1.096 | 1.096 | 1.096 | 0.04 |
| 16 | 1.129 | 1.129 | 1.130 | 1.129 | 1.128 | 1.129 | 0.05 |
| 17 | 1.156 | 1.156 | 1.156 | 1.156 | 1.155 | 1.155 | 0.04 |
| 18 | 1.190 | 1.190 | 1.190 | 1.190 | 1.190 | 1.189 | 0.04 |

2.10 Intermediate precision: Shenqi Fuzheng injections taken from the same batch were assayed according to the method described above, respectively, except for under the variable factors such as on different dates and by different analysts.

2.10.1 Different analysis dates: Shenqi Fuzheng injections taken from the same batch were prepared according to the method of preparing test sample solution on different dates, respectively, and then three of the test sample solution were injected in parallel, followed by extracting the characteristic peaks and marking the retention time. Peak 12 of astragaloside IV was taken as Peak S, and then the relative retention time of each of other characteristic peaks. The results showed that the RSD values of the relative retention time of each characteristic peak was all less than 1%, as shown in Table 6:

TABLE 6
Results of analysis on different dates

| | Date 1 | | | Date 2 | | | RSD (%) |
|---|---|---|---|---|---|---|---|
| 1 | 0.517 | 0.518 | 0.517 | 0.516 | 0.516 | 0.517 | 0.19 |
| 2 | 0.541 | 0.541 | 0.542 | 0.542 | 0.541 | 0.541 | 0.08 |
| 3 | 0.585 | 0.586 | 0.585 | 0.585 | 0.585 | 0.586 | 0.03 |
| 4 | 0.623 | 0.624 | 0.623 | 0.623 | 0.623 | 0.623 | 0.07 |
| 5 | 0.661 | 0.661 | 0.661 | 0.661 | 0.661 | 0.661 | 0.05 |
| 6 | 0.715 | 0.715 | 0.715 | 0.715 | 0.715 | 0.715 | 0.01 |
| 7 | 0.786 | 0.786 | 0.787 | 0.786 | 0.786 | 0.786 | 0.03 |
| 8 | 0.819 | 0.820 | 0.820 | 0.820 | 0.820 | 0.819 | 0.02 |
| 9 | 0.847 | 0.847 | 0.848 | 0.848 | 0.848 | 0.847 | 0.03 |
| 10 | 0.923 | 0.923 | 0.923 | 0.923 | 0.922 | 0.922 | 0.01 |
| 11 | 0.972 | 0.972 | 0.973 | 0.972 | 0.972 | 0.972 | 0.02 |
| S | 1.000 | 1.000 | 1.000 | 1.000 | 1.000 | 1.000 | 0.00 |
| 13 | 1.044 | 1.044 | 1.044 | 1.045 | 1.044 | 1.044 | 0.02 |
| 14 | 1.074 | 1.074 | 1.074 | 1.074 | 1.074 | 1.074 | 0.01 |
| 15 | 1.096 | 1.097 | 1.096 | 1.096 | 1.096 | 1.096 | 0.03 |
| 16 | 1.128 | 1.129 | 1.129 | 1.129 | 1.128 | 1.128 | 0.03 |
| 17 | 1.155 | 1.156 | 1.155 | 1.156 | 1.156 | 1.155 | 0.03 |
| 18 | 1.189 | 1.189 | 1.190 | 1.189 | 1.189 | 1.188 | 0.04 |

2.10.2 Different analysts: Shenqi Fuzheng injections taken from the same batch were prepared according to the method of preparing test sample solution by different analysts, respectively, and three of the test sample solution were injected in parallel, followed by extracting the characteristic peaks and marking the retention time. Peak 12 of astragaloside IV was taken as Peak S, and then the relative retention time of each of other characteristic peaks was calculated. The results showed that the RSD values of the relative retention time of each characteristic peak was all less than 1%, as shown in Table 7:

TABLE 7
Results of analysis by different analysts

| | Analyst 1 | | | Analyst 2 | | | RSD (%) |
|---|---|---|---|---|---|---|---|
| 1 | 0.516 | 0.518 | 0.519 | 0.519 | 0.519 | 0.519 | 0.18 |
| 2 | 0.542 | 0.544 | 0.544 | 0.544 | 0.543 | 0.544 | 0.16 |
| 3 | 0.586 | 0.588 | 0.588 | 0.589 | 0.586 | 0.587 | 0.18 |
| 4 | 0.623 | 0.625 | 0.625 | 0.626 | 0.624 | 0.625 | 0.16 |
| 5 | 0.661 | 0.663 | 0.663 | 0.663 | 0.661 | 0.663 | 0.17 |
| 6 | 0.714 | 0.716 | 0.717 | 0.717 | 0.714 | 0.716 | 0.19 |
| 7 | 0.786 | 0.788 | 0.789 | 0.789 | 0.785 | 0.788 | 0.19 |
| 8 | 0.819 | 0.821 | 0.822 | 0.822 | 0.819 | 0.821 | 0.15 |
| 9 | 0.848 | 0.848 | 0.849 | 0.849 | 0.847 | 0.848 | 0.07 |
| 10 | 0.922 | 0.922 | 0.923 | 0.923 | 0.922 | 0.923 | 0.03 |
| 11 | 0.972 | 0.972 | 0.972 | 0.972 | 0.972 | 0.972 | 0.03 |
| S | 1.000 | 1.000 | 1.000 | 1.000 | 1.000 | 1.000 | 0.00 |
| 13 | 1.044 | 1.044 | 1.044 | 1.044 | 1.044 | 1.044 | 0.02 |
| 14 | 1.074 | 1.074 | 1.074 | 1.074 | 1.074 | 1.074 | 0.01 |
| 15 | 1.096 | 1.095 | 1.095 | 1.095 | 1.096 | 1.096 | 0.02 |
| 16 | 1.128 | 1.129 | 1.129 | 1.128 | 1.128 | 1.128 | 0.02 |
| 17 | 1.155 | 1.155 | 1.155 | 1.155 | 1.155 | 1.156 | 0.03 |
| 18 | 1.189 | 1.188 | 1.188 | 1.188 | 1.189 | 1.189 | 0.04 |

### Example 2

### Identification of Shenqi Fuzheng Injection

In recent years, as the use of Shenqi Fuzheng injection is increasing in clinical, some criminals are motivated by economic interest and counterfeit Shenqi Fuzheng injection with other varieties for sale to make huge profits, which results in a great negative impact on the brands for Shenqi Fuzheng injection, and has caused significant economic loss to these enterprises which produce and sell Shenqi Fuzheng injections legally. These counterfeit Shenqi Fuzheng injections have almost the same appearance as the real ones, so it is hard to distinguish the real from the fake.

In this example, the method described in Example 1 was adopted to test the certified Shenqi Fuzheng injection (provide by Livzon Group Limin Pharmaceutical Factory), a suspected sample and a Danshen injection, so as to establish the corresponding fingerprint profiles. The results were shown in FIG. 4. It can be seen that the fingerprint profile of the suspected sample is completely different from that of the certified Shenqi Fuzheng injection, the components of the counterfeit were inferred by using the precise molecular weight provided by mass spectrometry. It was substantially confirmed that the components of the counterfeit were derived from Danshen.

Thus, the Shenqi Fuzheng injection fingerprint profile established by ultra high pressure liquid chromatography-mass spectrometer (UHPLC-MS) can be used to identify the authenticity of a Shenqi Fuzheng injection in a fast and accurate manner, and can also be used to analyze the counterfeit qualitatively and substantially confirm the source. If there is any counterfeit Shenqi Fuzheng injection with DanShen injection by criminals, it can be identified according to the method described above, i.e. comparing the fingerprint profile of the injection with that of the certified Shenqi Fuzheng injection, inferring the components of the counterfeit by using the precise molecular weight provided by mass spectrometry, substantially confirming the component source of the counterfeit.

Therefore, application of the Shenqi Fuzheng injection UHPLC-MS fingerprint profile can avoid counterfeiting, and ensure normal production and good circulation of the Shenqi Fuzheng injection so as to protect legitimate rights and interests of manufactures.

The method provided by the present invention for establishing Shenqi Fuzheng injection fingerprint profile was described in details hereinbefore. The principles and embodiments of the present invention are described herein with reference to some specific examples, however, the examples described above are only intended to help understand the method and the core idea of the present invention. It should be noted that, for those skilled in the art, a number of improvements and modifications can be introduced to the present invention, without departing from the principles of the present invention, and these improvements and modifications shall fall into the scope defined by the appended claims.

1. A method for establishing Shenqi Fuzheng injection fingerprint profile, comprising testing Shenqi Fuzheng injection by ultra-high pressure liquid chromatography-mass spectrometer, wherein the chromatographic conditions include the followings:
Chromatographic column: Agilent Zorbax Eclipse Plus C18, 2.1 mm×100 mm, 1.8 μm;
Mobile phase: Mobile phase A is 0.1% (v/v) formic acid aqueous solution, mobile phase B is 0.1% (v/v) formic acid acetonitrile solution;
Using gradient elution according to the following elution program, wherein the proportions of the mobile phases are all volume percentages:
0-5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%;
Preferably, the chromatographic conditions also include the followings:
Flow rate: 0.35 ml/min;
Column temperature: 40° C.;
Injection volume: 5 μl.

2. The method according to claim 1, wherein the mass spectrometry conditions include the followings:
The ion source is an ESI source, and detection is operated in negative ion mode;
Atomized gas pressure: 35 psig;
Dry gas temperature: 350° C.;
Dry gas flow rate: 10 L/min;
Capillary voltage: 3,500 V;
Voltage at capillary exit: 135 V.

3. The method according to claim 1, wherein said method further comprises preparation of control solutions by the following steps: accurately weighing an appropriate amount of calycosin glucoside or astragaloside IV, and adding methanol to prepare a solution containing 0.004 mg of calycosin glucoside per ml or 0.006 mg of astragaloside IV per ml, respectively.

4. The method according to claim 1, wherein the method further comprises preparation of a test sample solution by the following step: filter Shenqi Fuzheng injection through a 0.22 μm microporous filter membrane.

5. The method according to claim 1, wherein the method comprises the following steps:
(1) Preparation of control solution: Accurately weighing an appropriate amount of calycosin glucoside or astragaloside IV, and then adding methanol to prepare a solution containing 0.004 mg of calycosin glucoside per ml or 0.006 mg of astragaloside IV per ml, respectively;
(2) Preparation of test sample solution: Filtering Shenqi Fuzheng injection through a 0.22 μm microporous filter membrane;
(3) Determination: Accurately aspirating 5 μl of the control solution or the test sample solution, respectively, and then injecting the solutions into a ultra-high pressure liquid chromatography-mass spectrometer, conducting determination according to the following conditions to obtain the Shenqi Fuzheng injection fingerprint profile;
wherein the chromatographic conditions include the followings:
Chromatographic column: Agilent Zorbax Eclipse Plus C18, 2.1 mm×100 mm, 1.8 μm;
Mobile phase: mobile phase A is 0.1% (v/v) formic acid aqueous solution, mobile phase B is 0.1% (v/v) formic acid acetonitrile solution;
Using gradient elution according to the following elution program, wherein the proportions of the mobile phases are all volume percentages:
0-5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%;
Preferably, the chromatographic conditions also Include the followings:
Flow rate: 0.35 ml/min;
Column temperature: 40° C.;
Preferably, the mass spectrometry conditions include the followings:
The ion source is an ESI source, and detection is operated in negative ion mode;
Atomized gas pressure: 35 psig;
Dry gas temperature: 350° C.;
Dry gas flow rate: 10 L/min;
Capillary voltage: 3,500 V;
Voltage at capillary exit: 135 V.

6. The method according to claim 1, wherein the method further comprises: comparing multiple Shenqi Fuzheng injection fingerprint profiles, picking out common characteristic peaks to obtain the Shenqi Fuzheng injection characteristic fingerprint profile.

7. The method according to claim 1, wherein the Shenqi Fuzheng injection fingerprint profile or Shenqi Fuzheng injection characteristic fingerprint profile comprises 18 characteristic peaks, and the retention time of each characteristic peak is as follows:
Peak 1: 7.1 min, Peak 2: 7.5 min, Peak 3: 8.1 min, Peak 4: 8.6 min, Peak 5: 9.2 min, Peak 6: 9.9 min, Peak 7: 10.9 min, Peak 8: 11.3 min, Peak 9: 11.7 min, Peak 10: 12.7 min, Peak 11: 13.4 min, Peak 12: 13.7 min, Peak 13: 14.4 min, Peak 14: 14.8 min, Peak 15: 15.1 min, Peak 16: 15.5 min, Peak 17: 15.9 min, Peak 18: 16.3 min.

8. The method according to claim 1, wherein the Shenqi Fuzheng injection fingerprint profile or the Shenqi Fuzheng injection characteristic fingerprint profile takes the control astragaloside IV as a reference peak, by which the relative retention time of each characteristic peak is calculated, as follows:
Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, Peak 18: 1.19.

9. The method according to claim 1, wherein in the Shenqi Fuzheng injection fingerprint profile or the Shenqi Fuzheng injection characteristic fingerprint profile, Peak 1 and Peak 12 are calycosin glucoside and astragaloside IV, respectively; preferably wherein the ratio of the area of the calycosin glucoside peak and the astragaloside IV peak to the area of the corresponding reference peak is 0.5-1.5.

10. A method for identifying Shenqi Fuzheng injection, comprising comparing the fingerprint profile or the characteristic fingerprint profile of the test samples established according to the method of claim 1 with the standard fingerprint profile or the characteristic fingerprint profile established according to the method described above.
